(19) United States
(12) Patent Application Publication
(10) Pub. No.: US 2020/0155479 A1
(43) Pub. Date: May 21, 2020

(54) Method for infusing whole coffee beans and Tea Leaves with cannabis (Hemp) ingredients

(71) Applicant: Dawn Annette Hackney, Trinity, FL (US)

(72) Inventor: Dawn Annette Hackney, Trinity, FL (US)

(21) Appl. No.: 16/682,838

(22) Filed: Nov. 13, 2019

Related U.S. Application Data
(60) Provisional application No. 62/756,190, filed on Nov. 6, 2018.

(51) Int. Cl. A61K 31/05; A61K 36/74; A61K 47/14; A61K 36/82

(52) U.S. Cl. CPC A61K 31/05; A61K 36/82; A61K 47/14; A61K 36/74

(57) ABSTRACT

Methods of infusing whole coffee beans and tea leaves or tea blends containing cannabinoids that are extracted from Cannabis/Hemp plant. According to one embodiment, the method includes the steps of (a) extracting cannabinoids from the Cannabis/Hemp plant into an isolate powder form; and (b) infusing the isolate powder form cannabinoids into whole coffee beans or tea leaves/blends. The disclosed methods produce coffee and tea beverages that possess wellness benefits associated with coffee beans, tea and cannabinoids extracted from the Cannabis/Hemp plant. The claimed method of infusing coffee beans and tea leaves with CBD (Cannabidiol) that contains a consistent milligram of CBD (Cannabidiol) in each measurement of 5 mg to 100 mg as well as maintain a shelve life without the microbial bacteria growth. Further, the coffee bean can be ground to be used as espresso or traditional drip brew and the tea leaves can be steeped or chilled for human consumption.

### CROSS REFERENCE TO RELATED APPLICATION

The present application is related to U.S. Provisional Patent Application No. 62/756,190, filed Nov. 6, 2018, entitled “Method for infusing whole coffee beans and Tea Leaves with cannabis ingredients.” The present application hereby claims priority under 35 U.S.C. sctn.119(e) to U.S. Provisional Patent Application No. 62/756,190 and is hereby incorporated by reference into the present application as if fully set forth herein.

### STATEMENT REGARDING FEDERALLY SPONSORED RESEARCH AND DEVELOPMENT

No federal funds were used related to the discovery of this infusion method

### BACKGROUND OF THE INVENTION

As far back as 1000 BC Cannabis has been attributed as a wellness plant, whereby India developed a consumable beverage called Bhang which contained Milk and Cannabis used as an anesthetic and anti-phlegmatic. Hedge forward to the 21st Century, on Sep. 19, 2019 it was announced that nine research grants totally $3 million has been awarded to study the medicinal effects of Cannabidiol (CBD), a non-psychoactive cannabinoid of the Cannabis/Hemp plant. Cannabis, Hemp and Marijuana have been used interchangeable to describe the same plant. For our purposes, we will refer to the plant as Cannabis. The Cannabis plant has two distinct types; Cannabis sativa and Cannabis indica. Each Cannabis type has several different strains. The Cannabis plants produce a group of chemicals called cannabinoids. Agriculturalist have inter-bred each type in an effort to develop a new crop with desirable properties. Agriculturalist may inter-breed to develop a new crop that the desired outcome would be an increase in CBD (Cannabidiol) vs THC (Tetrahydrocannabinol).

Brewed beverages, such as coffee and teas, are among the most popular and common beverages that are consumed by people in varies cultures and countries around the world. Coffee and Tea in its natural state has been known to contain many health benefits from increased fiber intake to heart health. The present invention relates to enhancing whole coffee beans and tea leaves/blends through a proprietary infusion process containing CBD (Cannabidiol) isolate which is one of the cannabinoids of the Cannabis plant while maintaining it shelve stability and consistency of CBD (Cannabidiol) mg per serving. This infusion process would allow the benefits of coffee and tea to be collective with the health benefits of CBD (Cannabidiol) to further create an alternative natural medicine that can be used on a daily basis. The present inventor has researched the historical and technical solutions as it relates to this topic. Upon a detail review of varies literature sources associated with this subject matter, no conclusive evidence has been disclosed that relates to the current inventor's profiling, compounding and delivery of the present invention. For this reason, and as set forth and claimed below, the utilities of the present solution, as offered for consideration herein are respectfully proposed to constitute invention, as defined by statute.

### BRIEF SUMMARY OF THE INVENTION

The embodiment of the present invention addresses the need of combining the natural benefits of Tea and Coffee with the natural benefits of Cannabidiol extract from the Cannabis plant for human consumption while maintaining consistency of Cannabidiol milligrams in each measurement. This invention would allow the availability of CBD (Cannabidiol) infused Coffee and Tea Products to communities around the world. The consistent CBD (Cannabidiol) milligrams in each measurement, would allow consumers to control the intake of CBD milligrams based on their already used metric system. This proprietary infusion process uses CBD (Cannabidiol) extracted into an isolate powder form and homogenized with MCT (Medium Chain Triglycerides) as a carrier oil that is further infused into measured controllable batches of coffee beans and Tea Leaves. CBD (Cannabidiol) extracted into an isolate powder is 99.9%-100% CBD (Cannabidiol) represented in exhibit A through a lab report. Isolate CBD (Cannabidiol) powder has been used as an additive in food and beverage in its powder form. This addition to food and beverage does not allow for consistent milligrams in each measurement due to settlement of the powder as well as other factors. MCT (Medium Chain Triglycerides) is extracted from the Coconut or Palm Kernel Oil. MCT (Medium Chain Triglycerides) C6, C8, C10 and C12 are the main fatty acids found in MCT. Due to the shorter chain length, MCTs are more rapidly broken down and absorbed into the body creating a preferred carrier oil to deliver the CBD (Cannabidiol). Researchers believe that there are significant benefits from diets that are high in MCT-rich foods which may support healthy cholesterol levels, may improve brain function in people with Alzheimer's disease, may help reduce insulin resistance and other health benefits. However, the evidence is diverse and more research is needed.

There are different methods in producing a coffee product with consistent amount of THC or CBD. However, the research the inventor discovered did not use a natural carrier oil such as MCT or include a process to avoid bacteria growth.

The claimed method of infusing coffee beans and tea leaves with CBD (Cannabidiol) that contains a consistent milligram of CBD (Cannabidiol) in each measurement of 5 mg to 100 mg as well as maintain a shelve life without the microbial bacteria growth. Further, the coffee bean can be ground to be used as espresso or traditional drip brew and the tea leaves can be steeped or chilled for human consumption.

These and other characteristics of the disclosed embodiments will become more apparent from the following description and illustrative embodiments which are described in detail with reference to the accompanying drawings.

### BRIEF DESCRIPTION OF THE DRAWINGS

The drawings illustrate a sample embodiment of a set of process flow diagrams as it pertains to the current infusion process invention using Cannabidiol (CBD) Isolate.

FIG. 1 is an illustration of the infusion process flow diagram into whole coffee beans

FIG. 2 is an illustration of the infusion process flow diagram into a low absorption tea blend

FIG. 3 is an illustration of the infusion process flow diagram into high absorption tea leaves

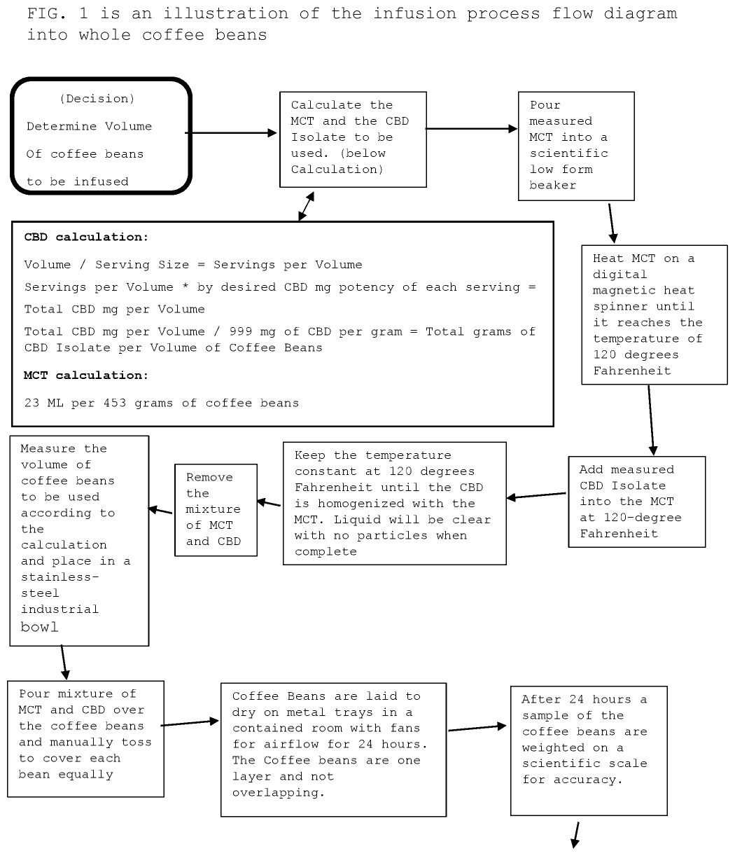

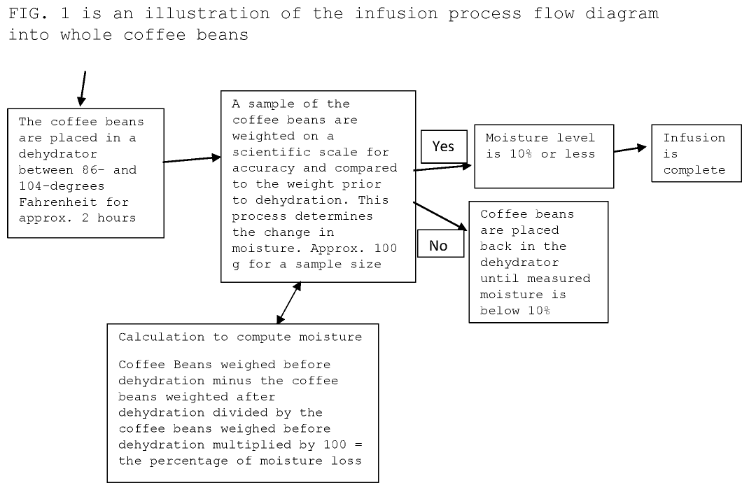

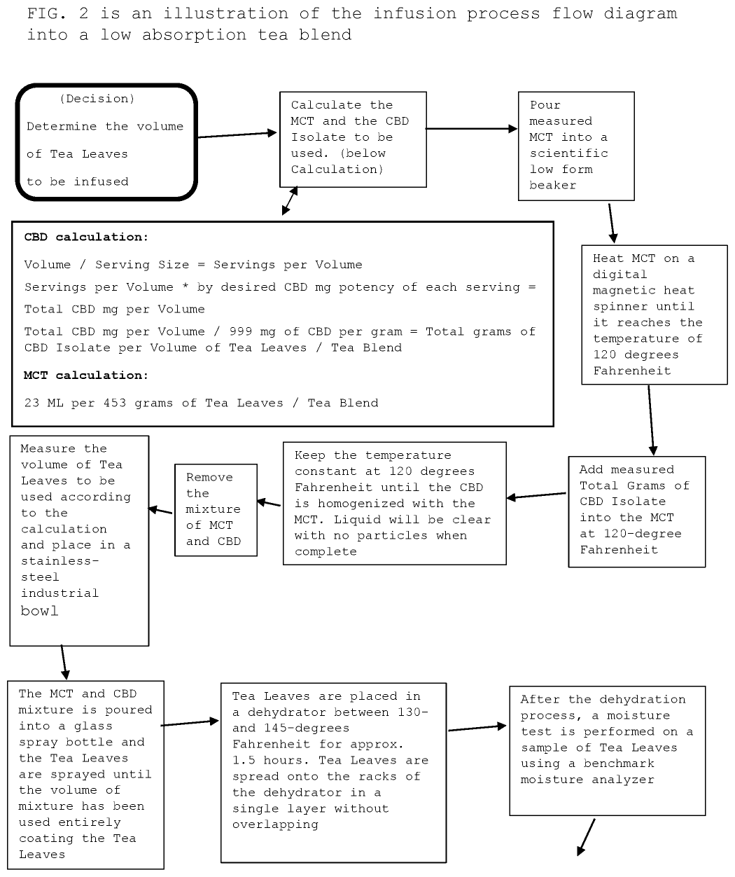

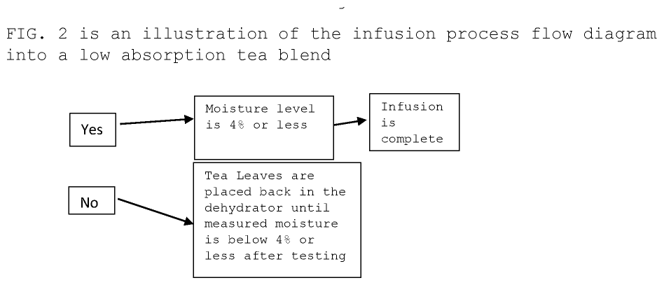

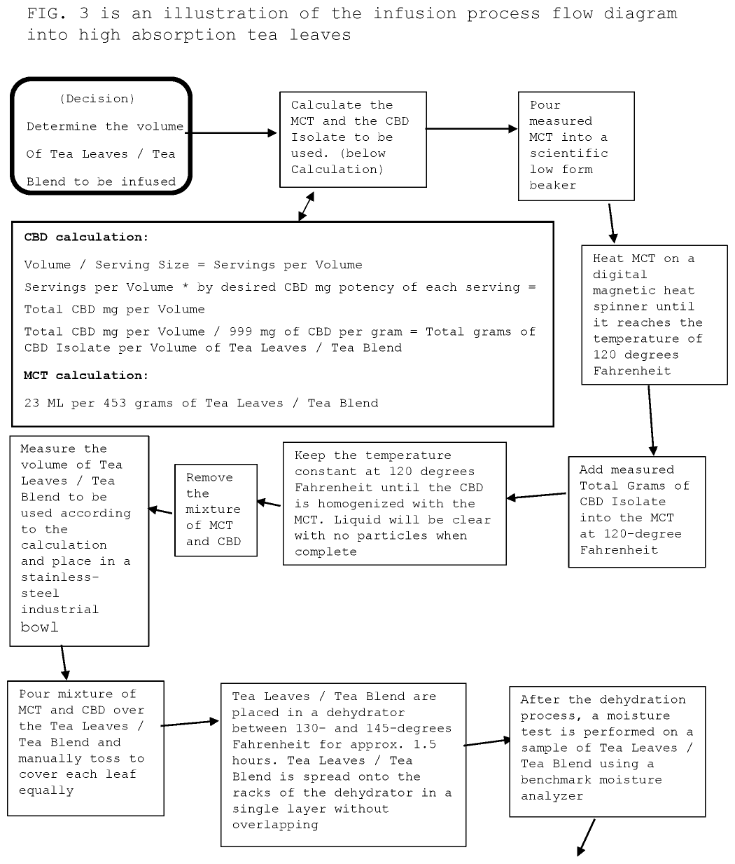

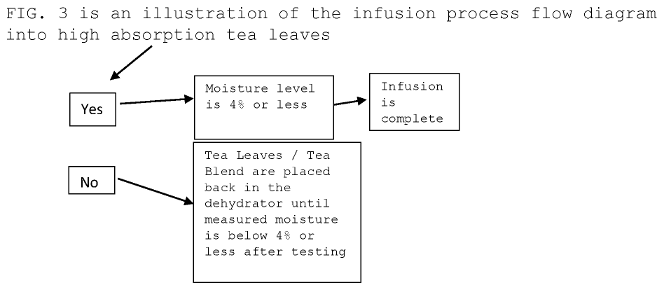

### DETAILED DESCRIPTION OF THE INVENTION

The present disclosure is directed to the inventive method of infusing coffee beans and tea leaves/tea blends containing cannabinoids that are extracted from the Cannabis plant. The disclosed method produces coffee beans and tea leaves/tea blends that possesses the benefits of both coffee or tea leaves and the active ingredients of the cannabinoids from the Cannabis plant. The infusion method can be used to produce consistent milligrams of cannabinoids per serving while maintaining a stable shelve life. The infused coffee beans can be ground to be used in a common drip method coffee pot or ground fine to be made into an espresso. The infused tea leaves/tea blend can be brewed hot or chilled. When consumed, the flavor of the coffee bean or tea leaves/tea blends will remain unchanged as MCT and CBD is flavorless.

### Definition of Terminology

Cannabis—is a genus of flowering plants in the family Cannabaceae. The number of species within the genus is disputed. Three species may be recognized: Cannabis sativa, Cannabis indica, and Cannabis ruderalis; C. ruderalis may be included within C. sativa; all three may be treated as subspecies of a single species, C. sativa;

or C. sativa may be accepted as a single undivided species. The genus is widely accepted as being indigenous to and originating from Central Asia, with some researchers also including upper South Asia in its origin. Reference https://en.wikipedia.org/wiki/Cannabis

Cannabinoids—any of a group of closely related compounds which include cannabinol and the active constituents of cannabis. Reference https://en.m.wikipedia.org/wiki/Cannabidiol

Cannabidiol (CBD)—is a naturally occurring compound found in the resinous flower of cannabis, a plant with a rich history as a medicine going back thousands of years. Today the therapeutic properties of CBD are being tested and confirmed by scientists and doctors around the world. A safe, non-addictive substance, CBD is one of more than a hundred phyto-cannabinoids which are unique to the cannabis plant and endow the plant with its robust therapeutic profile. Reference: https://www.projectcbd.org/cbd-101/what-is-cbd

CBD Isolate—CBD is the definition as state about that is manufactured via an extraction process. Once the concentrated CBD is extracted from the plant, it must purify for a period of time, with the chemical compounds needed to create CBD Isolate filtrated out of the cannabis that's extracted from the plant. Once the CBD is filtered out, the CBD oil removed from the cannabis plant goes through one last process, called winterization, where any remaining plant particles are removed from the CBD oil. Winterization is the process for chilling chemical compounds, which is needed to extract impurities from the CBD. With CBD Isolate, the winterization process takes away any waxes or other unneeded components (like terpenes, chlorophyll and/or flavonoids) remaining in the CBD. Once that process is complete, the manufacturer is left with pure CBD powder, which has no levels of THC and is at a level that is 99% pure cannabidiol. Reference https://www.thestreet.com/lifestyle/what-is-cbd-isolate-14986685

Infusion—The combination of MCT (Medium Chain Triglycerides) and Cannabidiol (CBD) isolate to prepare coffee beans or tea leaves/tea blends by soaking the leaves of the tea or tea blend with the combination.

Medium-chain triglycerides (MCTs)—are triglycerides with two or three fatty acids having an aliphatic tail of 6-12 carbon atoms. i.e C6, C8, C10 and C12. MCT is found in the coconut or palm plant. Reference https://en.wikipedia.org/wiki/Medium-chain_triglyceride

Turning our attention to the details of the invention. There are several different methods for extracting cannabinoids from the cannabis plant. The three most common are Alcohol, Hydrocarbon and Supercritical CO2 extraction methods. All of the methods produce an oil extracted from the cannabis plant that contain the cannabinoids. The oil is processed through a purifying filtration process to remove excess plant material. The oil is further refined to remove any impurities which is a process termed as winterization. After the winterization, if CBD isolate is desired, Chemists utilize a process called convergence chromatography to separate CBD from raw oil extract. This process is repeated until the cannabidiol is isolated in its most pure form—a fine white crystalline powder made up of 99.9% pure CBD. This invention is not focusing on the extraction process to obtain CBD isolate. The summary extraction process is merely to provide color to the process whereby CBD isolate is produced. This invention requires the use of CBD Isolate.

The CBD isolate or any other cannabinoid in a powder form are heated together with MCT at the ratios reflected in Table 1 below and temperatures between 110-120 degrees Fahrenheit to properly homogenize the two components. A digital heat magnetic spinner is used to homogenize the two components. The heat magnetic spinner allows the heat to remain constant as well as the speed of the spin. CBD Isolate is fat soluble. Therefore, combining the MCT oil and the CBD isolate at high temperatures and speed, the two components homogenizes equally throughout the mixture. The mixture is low in viscosity and therefore allows for consistency and ease of the application to the coffee bean or tea leaves/tea blend. Samples of the infused coffee beans and tea leaves is and has been tested in a certified lab to validate the potency and microbial as well as toxins.

The example below in Table 1 reflects the calculation that should be used to obtain the desired consistent CBD milligrams per serving. The below example uses 25 mg CBD as the desired milligrams per serving. The calculation is:

Volume/Serving Size=Servings per Volume

Servings per Volume*by desired CBD mg potency of each serving=Total CBD mg per Volume

Total CBD mg per Volume/999 mg of CBD per gram=Total grams of CBD Isolate per Volume of Coffee Beans or Tea Leaves being infused

TABLE 1
Coffee Beans

| | | Isolate at 99.9% | | |
|---|---|---|---|---|
| volume | MCT | CBD per MG | Serving Size | Potency |
| 453 grams | 23 ML | 1.126 grams | 2 Tablespoons or 10 grams | 25 mg per serving |

1 (453 grams volume divided by 10 grams serving size = 45 servings per volume)
2 (45 servings per volume multiplied by 25 mg CBD which is the desired CBD potency of each serving = 1125 mg of CBD)
3 (1125 mg of CBD in one pound or 453 grams of coffee beans if the desired CBD potency is 25 mg per serving)
4 (999 mg of CBD is in one gram of CBD Isolate)
5 (1125 total CBD mg divided by 999 mg of CBD per gram = 1.126 grams CBD is weighed and admixed with the MCT to achieve
the desired potency of 1125 mg CBD per pound or 453 grams)
Tea Leaves

| | | Isolate at 99.9% | | |
|---|---|---|---|---|
| volume | MCT | CBD | Serving Size | Potency |
| 453 grams | 23 ML | 1.126 grams | 2.8 grams of tea per serving | 25 mg per serving |

1 (453 grams volume divided by 2.8 grams serving size = 162 servings per volume)
2 (162 servings per volume multiplied by 25 mg which is the desired CBD potency of each serving = 4050 mg of CBD)
3 (4050 mg of CBD in one pound or 453 grams of tea leaves/tea blend if the desired CBD potency is 25 mg per serving)
4 (999 mg of CBD is in one gram of CBD Isolate)
5 (4050 total CBD mg divided by 999 mg of CBD = 4.05 grams CBD is weighed and admixed with the MCT to achieve
the desired potency of 4050 mg per pound or 453 grams)

The Food and Drug Administration (FDA) has classified Caprylic triglyceride which is an MCT “generally recognized as safe,” (GRAS) which means that it is acceptable for use in flavorings, drugs, and cosmetics, and as a direct food additive. Therefore, MCT to the extent necessary to dissolve the extracted cannabinoids can be in a final coffee or tea product.

The homogenized MCT and CBD Isolate mixture is poured over the top of the coffee beans that have been weighed to the volume represented in the calculation above in a stainless-steel industrial bowl. The coffee beans are hand tossed or a machine can be used that is specific to the bean turning process. This process allows each coffee bean to be coated with the mixture evenly. The coffee beans are spread out evenly on trays that are non-porous for 24 hours drying for absorption. The coffee beans are further placed in a dehydrator or a drying machine can be used at a temperature of 86 to 104 degrees Fahrenheit as to not compromise the quality of the coffee bean until the moisture level is 10% or less. The moisture level is measured by the internationally accepted standard method for determining moisture in coffee which is the loss of weight on heating method. This process prevents the growth of bacterial and mold. After the coffee beans have completed the above process, they can be ground or packaged as whole bean coffee to consumers.

The above finished product of coffee beans was independently certified lab tested and the desired CBD potency was achieved within a 10% deviation. Several samples from different batches of the infused coffee beans have been tested by the certified lab. These samples were infused with CBD milligrams from 20 to 100 MG. The microbial lab testing reflected no present of Aspergillus, Salmonella, or Escherichia.

The above process is the same for tea leaves/tea blends with the exception of the drying process as well as if the tea leaves are fragile or fine in texture, the tea is sprayed vs hand tossed or machine rotated. For tea leaves and tea blends that are hardy in texture, then the mixture can be hand rotated as the same as the coffee bean. If the tea leaves are fragile or fine, the homogenized MCT/CBD mixture is placed in a spray bottle and each leaf is coated via a spray device. The complete volume of the mixture is used to maintain potency levels compared to volume used. The tea leaves are then placed on racks in a dehydrator at a temperature between 130-145 degrees Fahrenheit until the moisture level measures less than 4% to preserve the quality of the tea and prevent microbial content. The moisture level is measured by a benchtop moisture analyzer. This process ensures that the moisture level is low to assist in the prevention of the growth of bacterial and mold to maintain shelve stability.

After the tea leaves/tea blend have completed the above process, they can be bagged or packaged loose.

The above finished product of tea leaves/tea blends was lab tested and the desired CBD potency was achieved within an 8% deviation. Multiple samples of the tea leaves/tea blends from different batches were independently certified lab tested. The sample batches tested ranged from 20-100 MG of CBD potency. The microbial lab test passed testing with no present of Aspergillus, Salmonella, or Escherichia for each sample batch.

Certified lab test ware performed without the dehydration process during the research and development phase. The results presented slight levels of Escherichia and deviations in potency levels. This communicated to the inventor that the moisture levels were higher than expected and not shelf stable. Adding the dehydration process after infusion corrected both the potency and microbial issue.

The flash point of 170 degrees Fahrenheit, CBD Isolate will evaporate and reduce potency. This is particular important upon brewing and steeping. The desired brewing and steeping heat levels proved to be optimal between 150-160 degrees Fahrenheit. At this temperature, the potency of the CBD MG remained consistent with less than a 4% deviation in MG. Additionally, samples were taken from the top of the coffee or tea and the middle of the coffee and tea. Each sample resulted in the same CBD MG.

The relatively small deviation (4%) suggests that the infusion process to consumer brewing and steeping can be expected to be reliably produce a consistent dose of CBD or similar cannabinoid using a purified powder form of the cannabinoid.

The steeped tea was further researched to determine if climate change to between 35 and 38 degrees Fahrenheit would affect the CBD MG potency. The CBD potency level reflected no change and therefore suggest that infused CBD tea can be refrigerated after the steeping process and consumed with consistent CBD potency.

The description has not attempted to exhaustively enumerate all possible variations. That alternate embodiments may not have been presented for a specific portion of the invention, and may result from a different combination of described portions, or that other non-described alternate embodiments may be available for a portion, is not to be considered a disclaimer of those alternate embodiments. It will be appreciated that many of those non-described embodiments are within the literal scope of the following claims, and others are equivalent.

1- Whole coffee beans infused with MCT (Medium Chain Triglycerides) and CBD (Cannabidiol) Isolate at 99.9% MG

2- Whole coffee beans infused with MCT (Medium Chain Triglycerides) and CBD (Cannabidiol) Isolate at 99.9% MG that can be brewed to provide a consumable liquid that contains from approximately 20 mg to 100 mg of CBD per serving consistently and maintain a shelf life without microbial bacteria growth. The 20 mg to 100 mg of CBD per serving is not the deviation but rather the desired mg per serving. Potency levels were tested at levels from 20 mg to 100 mg per serving. Example: Infused coffee beans to produce 30 mg of CBD consistently per serving while maintaining no microbial bacteria growth.

3- Tea leaves and Tea blends infused with MCT (Medium Chain Triglycerides) and CBD (Cannabidiol) Isolate at 99.9% MG

4- Tea leaves and Tea blends infused with MCT (Medium Chain Triglycerides) and CBD (Cannabidiol) Isolate at 99.9% MG that can be steeped to provide a consumable liquid that contains from approximately 20 mg to 100 mg of CBD per serving consistently and maintain a shelf life without microbial bacteria growth. The 20 mg to 100 mg of CBD per serving is not the deviation but rather the desired mg per serving. Potency levels were tested at levels from 20 mg to 100 mg per serving. Example: Infused tea leaves to produce 30 mg of CBD consistently per serving while maintaining no microbial bacteria growth.
